(19) United States
(12) Patent Application Publication
(10) Pub. No.: US 2010/0168022 A1
(43) Pub. Date: Jul. 1, 2010

(54) Use of In-Vitro Culture to Design or Test Personalized Treatment Regimens

(76) Inventor: Christopher J. Centeno, Westminster, CO (US)

Correspondence Address: SWANSON & BRATSCHUN, L.L.C., 8210 SOUTHPARK TERRACE, LITTLETON, CO, 80120, US

(21) Appl. No.: 12/636,214

(22) Filed: Dec. 11, 2009

Related U.S. Application Data
(60) Provisional application No. 61/121,819, filed on Dec. 11, 2008.

(51) Int. Cl. A61K 38/19; C12Q 1/02; C40B 30/06; A61K 38/18; A61K 38/22; G06G 7/60

(52) U.S. Cl. 514/12; 435/29; 506/10; 703/11

(57) ABSTRACT

Provided herein are methods of assessing responsiveness to an agent, methods of customizing treatments, methods of screening agents, and methods of treatment. Also provided are systems and kits for carrying out the methods contemplated herein. An exemplary aspect includes a method of assessing whether a subject having a condition, disorder, dysfunction, or disease would respond to treatment with an agent. The method comprises obtaining a stem cell sample from the subject; culturing the sample; exposing the sample to an agent; evaluating the sample in the presence of and in the absence of the agent to create a sample profile; and comparing the sample profile to a reference profile indicative of responsiveness to the agent. The similarity between the sample profile and the reference profile indicates the subject's responsiveness to the agent.

### TECHNICAL FIELD

The invention generally relates to methods of designing and testing personalized treatment regimens. More particularly, the invention relates to methods of assessing responsiveness to treatment with a particular agent for a particular individual or patient population.

### BACKGROUND

Pharmaceutical and nutraceutical industries spend billions of dollars world wide on research and development of new products, but very few of those products ever make it to the market. In fact, the pharmaceutical industry spends about 75% of the average cost per successful drug on researching drugs that are not successful.

Screening technologies used to predict success or failure of a prospective drug or nutraceutical are limited. Such technologies include in vitro cell based or in vivo animal based models, models which often inadequately represent the condition for which the drug is being considered.

### SUMMARY OF THE EMBODIMENTS

Provided herein are methods of assessing responsiveness to an agent in a particular individual or patient population, methods of customizing treatments for a particular individual or patient population, methods of screening agents, and methods of treatment. Also provided are systems and kits for carrying out the methods contemplated herein.

Aspects include a method of assessing whether a subject having a condition, disorder, dysfunction, or disease would respond to treatment with an agent. The method comprises obtaining a stem cell sample from the subject; culturing the sample; exposing the sample to an agent; evaluating the sample in the presence of and in the absence of the agent to create a sample profile; and comparing the sample profile to a reference profile indicative of responsiveness to the agent. The similarity between the sample profile and the reference profile indicates the subject's responsiveness to the agent.

Other aspects include a method of providing a customized treatment for a subject for a particular need. The method comprises obtaining a stem cell sample from the subject; culturing the sample; exposing the sample to an agent; evaluating the sample in the presence of and in the absence of the agent to create a first sample profile; comparing the first sample profile to a first reference profile indicative of non-responsiveness to the agent, wherein the similarity between the first sample profile and the first reference profile indicates the subject's non-responsiveness to the agent; designing a custom compound and exposing the sample to the custom compound; evaluating the sample in the presence of and in the absence of the custom compound to create a second sample profile; and comparing the second sample profile to a second reference profile indicative of responsiveness to the agent. The similarity between the second sample profile and the second reference profile indicates the subject's responsiveness to the custom compound. The custom compound can be provided to the subject as a custom treatment.

Further aspects include a method of screening a custom compound for efficacy, safety, or lack of adverse effect in a subject. The method comprises obtaining a stem cell sample from a subject; culturing the sample; exposing the sample to a custom compound; evaluating the sample in the presence of and in the absence of the custom compound to create a sample profile; and comparing the sample profile to a reference profile generated by an agent which exhibits efficacy, safety, or lack of adverse effect. The similarity between the sample profile and the reference profile indicates the efficacy, safety, or lack of adverse effect of the custom compound.

Other aspects include a method of treating a subject in need thereof. The method comprises obtaining a stem cell sample from the subject; culturing the sample; exposing the sample to an agent; evaluating the sample in the presence of and in the absence of the agent to create a sample profile; and comparing the sample profile to a reference profile indicative of responsiveness to the agent. The similarity between the sample profile and the reference profile indicates the subject's responsiveness to the agent. The subject is then treated with the agent.

Still further aspects include a method of treating a subject in need thereof. The method comprises obtaining a stem cell sample from the subject; culturing the sample; exposing the sample to an agent; evaluating the sample in the presence of and in the absence of the agent to create a first sample profile; comparing the first sample profile to a first reference profile indicative of non-responsiveness to the agent, wherein the similarity between the first sample profile and the first reference profile indicates the subject's non-responsiveness to the agent; designing a custom compound and exposing the sample to the custom compound; evaluating the sample in the presence of and in the absence of the custom compound to create a second sample profile; and comparing the second sample profile to a second reference profile indicative of responsiveness to the agent. The similarity between the second sample profile and the second reference profile indicates the subject's responsiveness to the custom compound. The custom compound is provided to the subject as a custom treatment.

Still other aspects provide a computer-aided system comprising: (a) a computer readable medium comprising computer-executable instructions configured to predict responsiveness of a subject to treatment with an agent; (b) input data from: (i) a subject profile; and (ii) a reference profile; and (c) an algorithm capable of comparing the data; wherein the system outputs a predicted responsiveness by the subject to the agent.

Further aspects provide kits useful in carrying out some aspects described herein. In some aspects the kit includes the materials necessary to harvest the stem cells from a subject, for example the tools and supplies necessary to harvest and culture stem cells. Kits can include vials, tissue culture flasks, culture medium, and other like laboratory materials necessary for the harvest and isolation of materials useful in practicing the methods described herein. Kits can include materials useful in preparing a subject profile, one or more reference profiles, and materials useful in comparing a subject profile with a reference profile.

Aspects herein include subject population treatment regimens wherein embodiments provide assessing responsiveness to treatment for a particular agent for a subject population, for example, a population of subjects having osteoporosis.

These and various features and advantages of the invention will be apparent from a reading of the following detailed description and a review of the appended claims.

### BRIEF DESCRIPTION OF THE DRAWING

FIG. 1 shows one aspect of the methods described herein portrayed in a flow diagram. Alternative embodiments and aspects are contemplated herein.

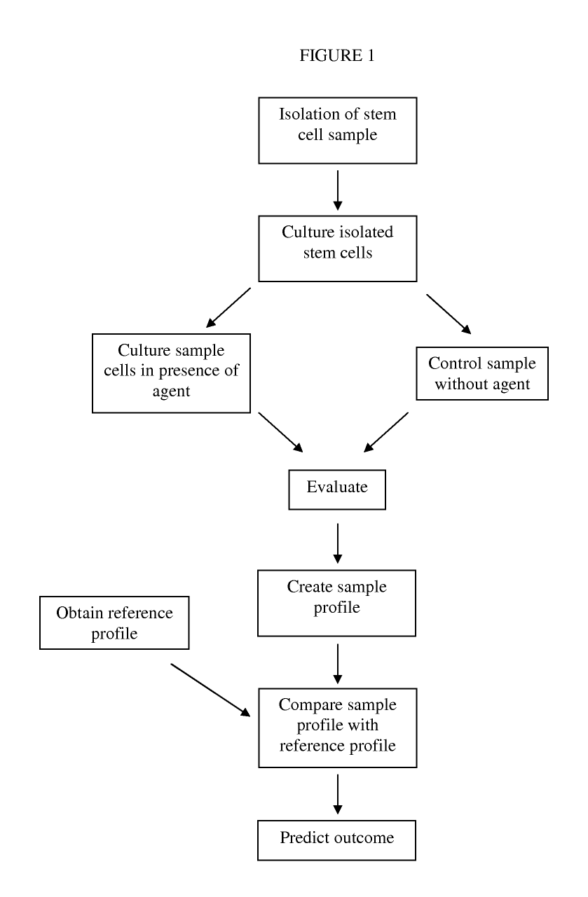

FIG. 2 provides a flow diagram illustrating the use of nucleated cells and FACS to design a custom supplement for a patient in need thereof.

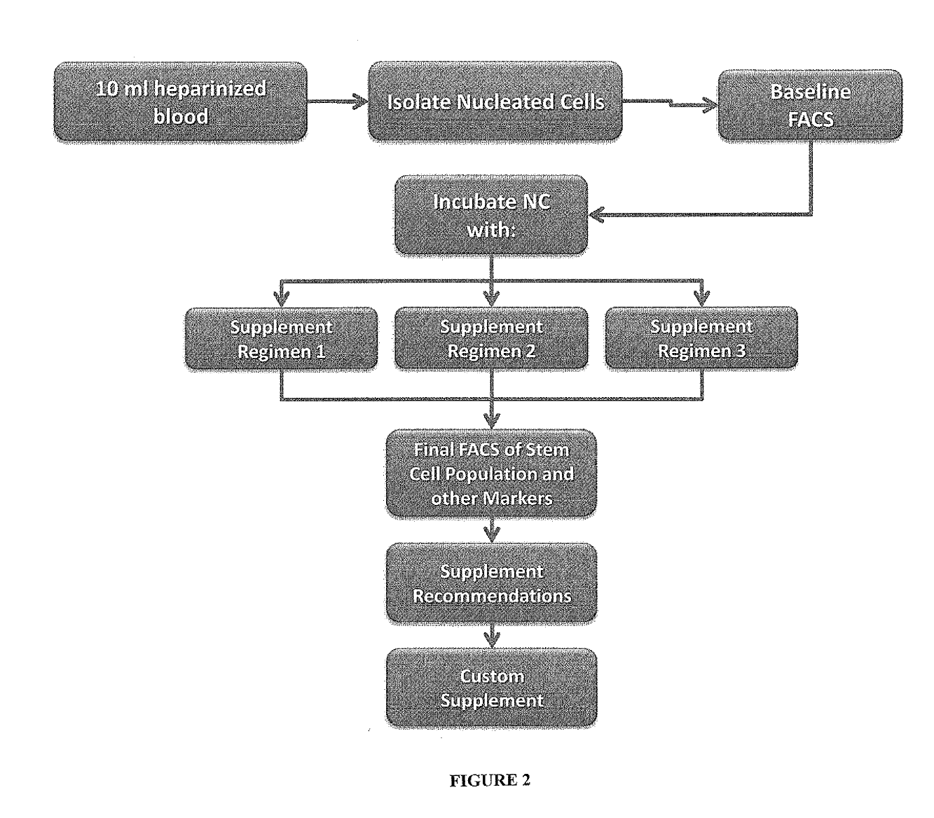

The Figures form part of the present specification and are included to further demonstrate certain aspects of the present invention. The invention may be better understood by reference to one or more of these drawings in combination with the detailed description of specific embodiments presented herein.

### DETAILED DESCRIPTION

Representative embodiments are provided below. While the invention will be described in conjunction with such embodiments, it will be understood that the invention is not intended to be limited to those embodiments. On the contrary, the invention is intended to cover all alternatives, modifications, and equivalents that may be included within the scope of the disclosure and any appended claims.

One skilled in the art will recognize many methods and materials similar or equivalent to those described herein can be used in and are within the scope of the practice of the present disclosure.

Unless described otherwise, technical and scientific terms used herein have the same meaning as commonly understood by one of ordinary skill in the art to which this disclosure belongs.

### DEFINITIONS

The following definitions are provided to facilitate understanding of certain terms used frequently herein and are not meant to limit the scope of the present disclosure.

As used herein, the singular forms “a”, “an”, and “the” include plural references, unless the content clearly dictates otherwise, and are used interchangeably with “at least one” and “one or more”.

“About” refers to a +/−10% variation from the nominal value. It is to be understood that such variation is always included in any given value provided herein, whether or not it is specifically identified.

As used herein, the terms “comprises”, “comprising”, “includes”, “including”, “contains”, “containing”, and any variations thereof, are intended to cover a non-exclusive inclusion, such that a system, device, process, method, etc. that comprises, includes, or contains an element or list of elements does not include only those elements but may include other elements not expressly listed or inherent to such system, composition, process, method, etc.

The phrase “in vivo response” refers to a clinical result in a patient due to exposure to an agent.

The phrase “cellular response” refers to a measurable in vitro response by a cell from exposure to an agent.

The term “profile” refers to a data set predictive of in vivo response.

The phrase “custom compound” refers to a compound, drug, or supplement that is designed to treat an individual or a specific patient population.

As used herein, the terms “subject” and “patient” are used interchangeably. It is contemplated that treatment based on the methods described herein may be under the care of a physician or other health care provider, thus treatment of a patient, or may be at the discretion of the individual, thus, treatment of the subject.

The term “agent” refers herein to any substance tested to evaluate an in vivo response using a reference profile. Examples include, but are not limited to, registered chemical entities, non-registered chemical entities, novel chemical entities, environmental reagents, nutraceuticals, and biologicals. An agent can also include a custom supplement, custom drug, or custom compound.

In some aspects, the agent is a growth factor, cytokine, hormone, or essential nutrient. Illustrative growth factors include transforming growth factor-beta, fibroblast growth factors, insulin like growth factors, bone morphogenic proteins; illustrative cytokines include cytokine-like 1 (Cyt-11); illustrative hormones include human growth hormone, and testosterone; and illustrative essential nutrients include ascorbic acid, pyruvate, and amino acids.

In other aspects, the agent is useful in treating skin, for example, agents such as alpha hydroxy acids, including glycolic acid and lactic acid, retinoic acid, vitamin C, azelaic acid, sodium sulfacetamide, topical metronidazole, oral or topical antibiotics, vitamin A or vitamin A analogs, benzoyl peroxide, etc.

A “known agent” is an agent which has been used in vivo or in vitro or which has been used to generate data.

### Aspects of the Invention

The ability to measure and correlate an in vitro cellular response with an in vivo response in a subject or a patient population has the potential to benefit humankind and commercial drug and pharmaceutical development. For example, ideal reference profiles could be generated for a large set of cellular responses to a particular agent, or for a large number of agents used to treat a particular condition, dysfunction, disorder, or disease. Drug discovery and design would benefit by making the entire process more efficient, for example, by providing and comparing reference profiles of agents typically used to treat a particular condition, dysfunction, disorder, or disease (and having a particular desired or undesired effect) to the profile of a new drug candidate in a particular individual or patient population.

The condition, disorder, dysfunction, or disease is contemplated herein. Illustratively, the condition, disorder, dysfunction, or disease is selected from the group consisting of: the degenerative conditions of aging selected from the group consisting of osteoarthritis, coronary heart disease, and osteoporosis; disorders of the immune system selected from the group consisting of lupus, systemic lupus erythematosus, multiple sclerosis, ankylosing spondylitis, scleroderma, and rheumatoid arthritis; disorders of the neurologic system selected from the group consisting of Alzheimer's disease, Parkinson's, and spinal cord injury; disorders of gastrointestinal system selected from the group consisting of irritable bowel syndrome, gastroesophageal reflux disease, Crohn's disease, and ulcerative colitis; and disorders of the musculoskeletal system selected from the group consisting of cartilage injuries, tendonopathies, fibromyalgia, ligament injuries, degenerative disc disease, and sciatica. In other embodiments, the condition, disorder, dysfunction, or disease affects the skin and includes, for example, acne, liver spots, actinic purpura, wrinkling of skin, sun damage, decrease in skin elasticity, etc.

Stem cells are the building blocks of the body. Supplements or medications that either positively or negatively impact these adult repair cells may have large long-term impacts on overall health. For example, cartilage homeostasis requires that mesenchymal stem cells differentiate into chondrocyte progenitors and then chondrocytes. See Alhadlaq, A. and J. J. Mao, Mesenchymal stem cells: isolation and therapeutics. Stem Cells Dev, 2004. 13(4): p. 436-48. A supplement that has a positive impact on this population may improve cartilage metabolism, while a prescription medication that has a negative impact may impair cartilage health. Recent reports of the negative impacts of quinolone antibiotics on collagen matrices exemplify these profound effects. See Barge-Caballero, E., et al., Quinolone-related Achilles tendinopathy in heart transplant patients: incidence and risk factors. J Heart Lung Transplant, 2008. 27(1): p. 46-51; Panigrahi, R., G. Longcroft-Wheaton, and K. Laji, Bilateral ankle pain and quinolone use: a case of tendon rupture secondary to quinolone use. Br J Hosp Med (Lond), 2008. 69(3): p. 168-9; Shortt, P., R. Wilson, and I. Erskine, Tendonitis: the Achilles heel of quinolones! Emerg Med J, 2006. 23(12): p. e63.

While many supplements have been promoted to have a positive impact on cartilage, claims regarding supplements and their effect on stem cells are based on limited in vitro data. For example, the benefit of glucosamine is based on in vitro data only. See Dudics, V., et al., Chondrogenic Potential of Mesenchymal Stem Cells from Patients with Rheumatoid Arthritis and Osteoarthritis: Measurements in a Microculture System. Cells Tissues Organs, 2008. The impact of prescription medications on stem cells is attracting more interest from researchers. For example, doxycycline can up regulate TGF-beta in peripheral joints thus leading to improved mesenchymal stem cell function. See Moutsatsos, I. K., et al., Exogenously regulated stem cell-mediated gene therapy for bone regeneration. Mol Ther, 2001. 3(4): p. 449-61. On the other hand, proton pump inhibitors may have a negative impact on mesenchymal stem cell function. However, supplements and medications are known to have widely different effects on any given subject based on that subject's medical history, genetic make-up, diet, environment, state of health (weight, blood pressure, fitness, etc.) and other like considerations. The inventors are unaware of any customized or personalized treatment regimen for a subject or subject population.

Thus, provided herein are methods of assessing responsiveness to an agent, methods of customizing treatments, methods of screening agents, and methods of treatment. Also provided are systems and kits for carrying out the methods contemplated herein.

Aspects include a method of assessing whether a subject having a condition, disorder, dysfunction, or disease would respond to treatment with an agent. The method comprises obtaining a stem cell sample from the subject; culturing the sample; exposing the sample to an agent; evaluating the sample in the presence of and in the absence of the agent to create a sample profile; and comparing the sample profile to a reference profile indicative of responsiveness to the agent. The similarity between the sample profile and the reference profile indicates the subject's responsiveness to the agent. This comparison facilitates which agents are most useful in treating that subject's condition, disorder, dysfunction, or disease.

Other aspects include a method of providing a custom treatment for a subject. The method comprises obtaining a stem cell sample from the subject; culturing the sample; exposing the sample to an agent; evaluating the sample in the presence of and in the absence of the agent to create a first sample profile; comparing the first sample profile to a first reference profile indicative of non-responsiveness to the agent, wherein the similarity between the first sample profile and the first reference profile indicates the subject's non-responsiveness to the agent; designing a custom compound treatment schedule and exposing the sample to the custom compound based on this schedule; evaluating the sample in the presence of and in the absence of the custom compound to create a second sample profile; and comparing the second sample profile to a second reference profile indicative of responsiveness to the agent. The similarity between the second sample profile and the second reference profile indicates the subject's responsiveness to the custom compound. The custom compound can be provided to the subject as a custom treatment.

Further aspects herein include a method of screening a custom compound for efficacy, safety, or lack of adverse effect. The method comprises obtaining a stem cell sample from a subject; culturing the sample; exposing the sample to a custom compound; evaluating the sample in the presence of and in the absence of the custom compound to create a sample profile; and comparing the sample profile to a reference profile generated by an agent which exhibits efficacy, safety, or lack of adverse effect. The similarity between the sample profile and the reference profile indicates the efficacy, safety, or lack of adverse effect of the custom compound.

Other aspects include a method of treating a subject in need thereof. The method comprises obtaining a stem cell sample from the subject; culturing the sample; exposing the sample to an agent; evaluating the sample in the presence of and in the absence of the agent to create a sample profile; and comparing the sample profile to a reference profile indicative of responsiveness to the agent. The similarity between the sample profile and the reference profile indicates the subject's responsiveness to the agent. The subject is then treated with the agent.

A “need” may or may not be associated with a condition, disorder, dysfunction, or disease, and includes mere desires. Exemplary needs include a need for increased muscle mass, or a desire to improve the quality of one's skin, mood, kidney function, liver function, etc.

Illustratively, treatment of a stem cell culture with chondroitin and/or hyaluronic acid, diluted to a physiologically appropriate concentration, for example about 2 to about 5 mg/ml in the stem cell culture medium, can be used to generate a sample profile. If a subject profile is generated which matches a reference profile indicative of responsiveness to chondroitin and/or hyaluronic acid, the chondroitin and/or hyaluronic acid is then administered to the patient to improve cartilage growth or cartilage maintenance. Similarly, treatment of a stem cell culture with glucosamine and/or collagen, diluted to a physiologically appropriate concentration, for example, about 500 to about 900 ng/ml in the stem cell culture medium, can be used to generate a sample profile. If a subject profile is generated which matches a reference profile indicative of responsiveness to glucosamine and/or collagen, the glucosamine and/or collagen is then administered to the patient to improve cartilage growth or cartilage maintenance. Likewise, treatment of a stem cell culture with one or more NSAIDS (non-steroidal anti-inflammatory drugs, for example, ketoprofen), diluted to a physiologically appropriate concentration, for example, about 10 to about 20 ng/ml in the stem cell culture medium, can be used to generate a sample profile. If a subject profile is generated which matches a reference profile indicative of responsiveness to one or more NSAIDS, the one or more NSAIDS are then administered to the patient to improve cartilage growth or cartilage maintenance.

Still further aspects include a method of treating a subject in need thereof. The method comprises obtaining a stem cell sample from the subject; culturing the sample; exposing the sample to an agent; evaluating the sample in the presence of and in the absence of the agent to create a first sample profile; comparing the first sample profile to a first reference profile indicative of non-responsiveness to the agent, wherein the similarity between the first sample profile and the first reference profile indicates the subject's non-responsiveness to the agent; designing a custom compound and exposing the sample to the custom compound; evaluating the sample in the presence of and in the absence of the custom compound to create a second sample profile; and comparing the second sample profile to a second reference profile indicative of responsiveness to the agent. The similarity between the second sample profile and the second reference profile indicates the subject's responsiveness to the custom compound. The custom compound is provided to the subject as a custom treatment.

Illustratively, nucleated cells are isolated from heparinized blood and a baseline FACS performed to characterize the cells. The cells are then split into groups and incubated with supplement regimens or custom compounds. For example, the cells are split into 4 groups, one control group and three different treatment groups. After an appropriate incubation time, the cells are subjected to a further FACS analysis. The difference between the baseline FACS and the FACS of the treatment groups provides the caregiver or patient with information useful in choosing a supplement regimen or custom compound.

### Isolation and Culture of Stem Cell Samples

Stem cells can be isolated according to any method known by one of skill in the art. Such methods are described in U.S. 2004/0229786, U.S. Pat. No. 6,200,606, U.S. Pat. No. 6,872,567 and WO 2007/087519, each of which is incorporated by reference in its entirety.

All stem cells are useful according to the methods described herein. For example, stem cell samples can comprise mesenchymal stem cells, epidermal stem cells, endodermal stem cells, or very small embryonic-like stem cells (VSEL; see Kucia et al., Developmental Dynamics, 2007, 236: 3309-3320 and Kucia et al., Cell Tissue Res., 2008, 331: 125-134, each of which is incorporated by reference in its entirety). Other cell types are contemplated herein, such as, for example, progenitor cells, including but not limited to epithelial progenitor cells.

Cells that have been manipulated to express certain proteins, including but not limited to proteins such as TGF-beta, FGF, VEGF, IGF, etc., are also contemplated for use according to the methods described herein.

Cultured cells are plated at an appropriate density and placed at about 37° C. in a 5% CO₂ incubator. As necessary, media is changed and cells are divided to maintain a desired density in the culturing flask or plate. In some aspects, fetal bovine serum and various recombinant growth factors are used to supplement the media. In other aspects, additives such as platelet lysate can be added to the culture medium. Exemplary methods and compositions for expansion of stem cell isolates are described in U.S. application Ser. No. 11/773,774 filed Jul. 5, 2007 and published as US2009/0010896, incorporated by reference herein in its entirety.

The type of cells harvested or targeted from a subject is based on supplements or medicaments to be tested. For example, where cartilage enhancing agents are to be tested, mesenchymal stem cells are harvested; where immunity enhancing agents are of interest, hematopoietic stem cells are targeted.

### Exposing a Stem Cell Sample to an Agent

Cultured stem cells can be treated with an agent according to methods known by those skilled in the art. The agent can be used at physiologically relevant concentrations, or in a range of concentrations in different culture flasks or wells. Control stem cell cultures maintained under the same conditions but without exposure to the agent provide baseline data.

After an appropriate period of exposure time, the cultures are evaluated to assess the response to an agent. Such response at the cellular level is considered a “cellular response”. Samples can be evaluated in the presence of and in the absence of the agent.

Examples of cellular response include gene expression, transcription, protein activation, protein inactivation, proliferation, differentiation, apoptosis, necrosis, cell cycle alterations, mitotic aberrations, alteration in morphology, membrane perturbations, change in motility, chemotaxis, contraction, relaxation, biosynthesis, secretion of signaling molecules, depolarization, repolarization, degranulation, adhesion, aggregation, change in metabolic rate, and other immediate cellular responses.

Cellular responses are generated by isolating stem cells from a subject, culturing the stem cells, and exposing the stem cells to an agent. In general, cellular responses can be assessed by measuring cell numbers, gene expression, protein abundances, protein activities, or any combination thereof. As such, cellular responses can be measured by any appropriate method known to those of skill in the art, including, for example, microarray, Western blot, Southern blot, Northern blot, light microscopy, electron microscopy, confocal microscopy, fluorescence microscopy, fluorescent plate reader, FACS, and a host of other methods. Further, to create sample profiles and reference profiles, more than one of the above methods can be used. In some aspects, only those cellular responses that are significant are selected. In other aspects, a clustering algorithm or other pattern recognition procedure is implemented to group the cellular responses into profile sets.

Exposure conditions are typically performed under recognized sterile culture conditions. Iteration of stem cell exposure to an agent can be from several times a day to once every day or more. Depending on the agent being tested, response to the agent can be measured once, every day for several days, or multiple times a day.

In some aspects, multiple responses can be considered for any one or more agents being treated, for example, cellular proliferation and differentiation.

### Reference Profile

A reference profile is a standard by which a subject profile can be compared. The reference profile can provide objective criteria and can identify agents that should be considered for a customized supplement or medication for a subject or subject population. In some embodiments, the reference profile is obtained by correlating in vitro data generated by treating stem cells gathered from one or more subjects with an agent to in vivo data obtained by treating the same subject(s) with the agent. The reference profile can contain clinical outcomes generated by treating a subject with an agent typically used to treat a certain condition, disease, disorder, or dysfunction. Each outcome is accompanied by in vitro data gathered from stem cells obtained from the same subject(s), or is some cases in vitro data gathered from stem cells obtained from different subject(s). In other embodiments, correlation of in vitro data and in vivo data is either unnecessary or not possible, and in such instances, the reference profile is simply in vitro data generated by treating stem cells gathered from one or more subjects with an agent. This embodiment can include utilizing in vitro data generated by treating stem cells gathered from a subject with a number of agents and at one or more treatment concentrations and comparing the results to identify the best agent and/or agent concentration for a particular cellular response.

A relevant reference profile can depend upon the condition of interest and/or the markers for efficacy or adverse effects being measured. Thus, certain in vitro responses can be correlated with certain in vivo responses, providing the basis for prediction of a response to an agent in a subject having the relevant condition, disorder, disease, or dysfunction, but never having had previous exposure to the agent.

Reference profiles can be pre-existing or produced using the methods described herein. For example, the reference profile can be obtained from a prospective study in which both the in vitro and in vivo data have yet to be collected. In other aspects, the reference profile can be obtained from a retrospective study in which the in vivo data is already available, but the in vitro data needs to be generated. A retrospective study would be appropriate in circumstances where exposure to the agent caused life-threatening events in some subjects; exposure of the same agent to other subjects would then likely be unethical (for example).

In some embodiments, a reference profile is indicative of a subject's non-responsiveness to an agent. In other embodiments, a reference profile is indicative of a subject's responsiveness to an agent. In still other embodiments, a reference profile is indicative of the efficacy, safety, or lack of adverse effect of an agent. In further embodiments, a reference profile is indicative of an adverse effect caused by an agent. In still further embodiments, a reference profile is indicative of an agent which exhibits undesired efficacy or is unsafe.

Reference profiles can also be generated for target subject populations, for example, a reference profile used for a population having osteoporosis where in vivo and in vitro data is tabulated/correlated for the subject population. The comparison to this type of profile can then be used to identify agents useful in the subject population.

### Sample Profile

A sample profile contains cellular response data obtained by isolating stem cells from a subject and exposing the stem cell sample to an agent of interest. To generate a sample profile, it can be helpful in some instances to identify cellular responses relevant to a specific condition, dysfunction, disorder, or disease. For example, for a particular condition, one skilled in the art might be aware of one, two, or three cellular responses which correlate with an adverse response to a particular agent, and thus, the evaluation of the sample in the presence of and in the absence of the agent would include performing a measurement of the one, two, or three cellular responses to generate the sample profile. In other instances, such relevant cellular responses can be unknown or can fail to accurately correlate with a predicted in vivo response, and as such, a broader analysis of cellular responses can be desirable. For example, the subject profile can comprise microarray data from several hundred genes or several thousand genes, any one or more of which can be indicative of a relevant cellular response.

### Comparison of Profiles and Outcome Prediction

Comparison of a sample profile to an appropriate reference profile can be performed manually or can be performed using a system, for example, a computer. The comparison step can be performed mentally, without physically placing the two profiles near each other. For example, the individual performing the comparison step can place the reference profile into his memory and make a mental comparison of profiles after obtaining the subject profile.

Likewise, predicting the outcome of a subject following treatment with an agent can be performed manually or can be performed using a system. Similar to the comparison step, the step of predicting the outcome can be performed mentally.

Parameters for comparison are discussed herein, but comparison can result in identification of one or more agents to customize, for example, a supplement for a particular usage in a subject.

### Methods

In one embodiment, stem cells are isolated from a patient with a known disease. The sample cells are cultured, and exposed to an agent. RNA extraction is performed, and the RNA is converted into a DNA library. This DNA library is subtracted from another DNA library obtained in a similar manner but using cells not exposed to the same agent. The resulting DNA contains those genes that, in response to the agent, are up regulated or down regulated (differential gene expression) and these genes are used to generate the subject profile. The subject profile is then compared to an appropriate reference profile associated with a positive outcome for the same disease. If the in vitro response correlates with a positive response in the reference profile, then the agent is more likely to cause the desired outcome in the subject.

In some embodiments, a series of agents are tested in this manner and the agent providing the closest or most appropriate results compared to the reference profile is identified as the agent of choice in treating the subject.

For example, ten supplements known in the literature to have an effect on a particular condition, for example, osteoarthritis, are tested in vitro using stem cells obtained from the subject. The supplement most highly associated with differentiation to chondrocytes and/or cellular proliferation would be recommended to the subject for treatment of osteoarthritis.

In one aspect, the subject profile and reference profile(s) can be generated using FACS to characterize the cells. Cells can be characterized many different ways using FACS, including but not limited to, detection of surface markers, viability determination, and stage of cell cycle. Other methods of characterizing cell populations and cellular responses are contemplated herein.

In another embodiment, the agent is a novel drug that has yet to be used in the context of a particular condition, disorder, disease, or dysfunction. The agent is screened for its ability to produce a cellular response that correlates with a positive clinical outcome.

In a further embodiment, the agent is known but is used to treat a different condition, disorder, dysfunction, or disease. The agent is screened for its ability to produce a cellular response that correlates with a positive clinical outcome.

In yet another embodiment, stem cells are isolated from subjects with the same condition, dysfunction, disorder, or disease but treated with a different agent. Differential gene expression is determined for a stem cell sample from each subject relative to stem cell samples from one or more untreated subjects. Alternatively, FACS can be used to characterize the stem cell sample from each subject relative to the stem cell samples from the untreated subjects. Data is collected on the in vivo response of the subjects to the agent and a set of reference profiles are generated that correlate the in vivo responses with each agent. The reference profile set can be used to create cellular response profiles predictive for in vivo results in patients with a particular condition, dysfunction, disorder, or disease.

### Custom Drug Design or Custom Treatment Regimen

In some embodiments, methods described herein are used in combination with custom treatment regimens or custom drugs. A sample profile obtained from a subject in need of treatment can be used to identify one or more traits to which a custom supplement or custom drug, or a custom treatment regimen can be designed.

A custom supplement, custom drug, or drug regimen can be created based on the desired outcome. As an example, if the desired outcome is stem cell chondrogenesis, any supplement and/or drug tested at doses that promote chondrogenic surface antigens on cells, up-regulate DNA/RNA expression that is associated with chondrogenesis, and/or promote actual chondrogenesis in vitro, can be used to treat a patient with osteoarthritis.

### Formulations

Agents and/or custom compounds identified for use in embodiments described herein can be formulated in any manner sufficient to treat a subject (or subject population), for example, formulated with a pharmaceutically acceptable carrier.

When used herein, the phrase “pharmaceutically acceptable carrier” refers to media generally accepted in the art for the delivery of biologically active agents to patients, in particular, mammals. Pharmaceutically acceptable carriers are typically formulated according to a number of factors well within the purview of those of ordinary skill in the art. These include, without limitation: the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 17th ed., Mack Publishing Company, Easton, Pa., 1985, which is incorporated herein by reference in its entirety.

Preparation of custom compounds or agents used according to the methods described herein includes addition of inert, solid or liquid pharmaceutically acceptable carriers. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, silica, sucrose, lactose, starch, or cellulose derivatives. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa., incorporated herein by reference for these uses.

Liquid form preparations include solutions, suspensions and emulsions. As an example, water or water-propylene glycol solutions for parenteral injection or addition of sweeteners and opacifiers for oral solutions, suspensions and emulsions, can be used. Liquid form preparations may also include solutions for intranasal administration. Gelatin capsules can be used to contain the active ingredient and a suitable carrier such as, but not limited to, lactose, starch, magnesium stearate, steric acid, or cellulose derivatives. Similar diluents can be used to make compressed tablets. Both tablets and capsules can be manufactured as sustained release products to provide for continuous release of medication over a period of time. Compressed tablets can be sugar-coated or film-coated to mask any unpleasant taste, or used to protect the active ingredients from the atmosphere, or to allow selective disintegration of the tablet in the gastrointestinal tract. Liquid dose forms for oral administration can also contain coloring or flavoring agents to increase patient acceptance.

Typically, water, pharmaceutically acceptable oils, saline, aqueous dextrose, and related sugar solutions and glycols, such as propylene glycol or polyethylene glycol, are suitable carriers for parenteral solutions. Solutions for parenteral administration can contain, for example, a water soluble salt of the active ingredient and suitable stabilizing agent(s). Antioxidizing agents, such as sodium bisulfite, sodium sulfite, or ascorbic acid, either alone or in combination, can act as suitable stabilizing agents. Also suitable as stabilizing agents are citric acid and its salts, and EDTA. In addition, parenteral solutions can contain preservatives such as, for example, benzalkonium chloride, methyl- or propyl-paraben, and chlorobutanol.

Oral compositions generally include an inert diluent or an edible carrier and may be compressed into tablets or enclosed in gelatin capsules. For purposes of oral therapeutic administration, the active agent or custom agent can be incorporated with excipients and used in the form of tablets, capsules, lozenges, or troches. Pharmaceutically compatible binding agents and adjuvant materials can be included as part of the composition. The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The agents or custom compounds can also be administered as a component of an elixir, suspension, syrup, wafer, chewing gum or the like. A syrup may contain, in addition to the active compounds, sucrose as a sweetening agent and certain preservatives, dyes, colorings, and flavors. The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenternal preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known, for example, as described in U.S. Pat. No. 4,522,811, which is incorporated by reference herein.

The agents or custom compounds can be prepared with carriers that protect the compound against rapid elimination from the body, such as time-release formulations or coatings such as enteric coatings to protect the compounds of the present invention from the acidic environment of the stomach. Enteric coated tablets are well known to those skilled in the art. In addition, capsules filled with small spheres, each coated to protect from the acidic stomach, are also well known to those skilled in the art. Other such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The agents and custom compounds used according to the methods described herein can be administered orally, parenterally (IV, IM, depo-IM, SQ, and depo-SQ), sublingually, intranasally (inhalation), intrathecally, topically, or rectally. Dosage forms known to those skilled in the art are suitable for delivery of the agents and custom compounds.

When administered orally, agents and custom compounds used according to the invention can be administered in usual dosage forms for oral administration as is well known to those skilled in the art and are described more fully herein. These dosage forms include the usual solid unit dosage forms of tablets and capsules as well as liquid dosage forms such as solutions, suspensions, and elixirs. When the solid dosage forms are used, it can be desirable that they are of the sustained release type so that the compounds of the invention need to be administered only once or twice daily.

Agents and custom compounds used according to the methods described herein can be administered sublingually.

Agents and custom compounds used according to the methods described herein can also be administered intranasally. When given by this route, the appropriate dosage forms are a nasal spray or dry powder, as is known to those skilled in the art.

Agents and custom compounds used according to the methods described herein can be administered intrathecally.

Agents and custom compounds used according to the methods described herein can be administered topically. When given by this route, the appropriate dosage form is a cosmetic, cream, ointment, or patch.

Agents and custom compounds used according to the methods described herein can be administered rectally by suppository as is known to those skilled in the art.

Agents and custom compounds used according to the methods described herein can be administered by implants as is known to those skilled in the art.

Given a particular agent or custom compound and a desired dosage form, one skilled in the art would know how to prepare and administer the appropriate dosage form.

The agent or custom compound is typically included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

Initial treatment regimens herein may also take into account in vitro sample profiles that included concentration parameters so an agent that demonstrated a favorable cellular response but only at higher concentrations may be administered to the subject in this context, i.e., higher dose of greater number of applications over a set period of time.

### Systems

The methods described herein can be executed manually or on automated systems, for example, a computer system capable of performing such methods. Thus, in some embodiments, a computer system comprising a computer usable medium containing code is useful herein.

In some embodiments, a computer-aided system is provided comprising: (a) a computer readable medium comprising computer-executable instructions configured to predict responsiveness of a subject to treatment with an agent; (b) input data from: (i) a subject profile; and (ii) a reference profile; and (c) an algorithm capable of comparing the data; wherein the system outputs a predicted responsiveness by the subject to the agent.

### Kits

In some embodiments, kits are provided which can be useful in carrying out the above-described methods. In some aspects, a kit is provided for assessing responsiveness of a subject to an agent potentially useful in treating a particular condition, dysfunction, disorder, or disease. In another aspect, a kit is provided for assessing the efficacy, safety, or adverse effect of an agent on a subject. Such kits can include, without limitation, tools and/or instructions for isolating stem cells, materials and/or protocols for culturing stem cells, one or more reference profiles or reference profile sets, instructions and/or examples for generating sample profiles, instructions and/or examples for comparing a sample profile with a reference profile, and instructions for assessing similarity between a sample profile and a reference profile. Instructions, protocols, and/or reference profiles can be provided in the form of books, pamphlets, or software, or can be available through the World Wide Web. The software can be included with the kit or can be in downloadable form from the internet.

The custom compounds or other agents can be enclosed in multiple or single dose containers, and can be provided in kits, for example, including component parts that can be assembled for use. For example, a custom compound in lyophilized form, and a suitable diluent, may be provided as separated components for combination prior to use. A kit may include a custom compound and a second therapeutic agent for co-administration. The custom compound and second therapeutic agent may be provided as separate component parts.

### EXAMPLES

The following examples are provided for illustrative purposes only and are not intended to limit the scope of the invention.

### Example 1

Synovial mesenchymal stem cells are isolated from a patient and cultured in an incubator at 37° C. and 5% CO₂. Each of chondroitin and/or hyaluronic acid is diluted to a physiologically appropriate concentration, about 2 to about 5 mg/ml, in the stem cell culture medium. After culturing the stem cell samples in the presence or absence of each agent, FACS is used to determine if the cells cultured in the presence of any one of the agents were being moved toward chondrogenesis. Exemplary markers used to make this determination include CD90⁻, CXCL1⁺, F1T3L⁺, CCR3⁺, and CCR4⁺. The expression levels of the various markers are used to create a sample profile. The sample profile generated by treatment of the stem cells is compared to a reference profile, or a profile of desirable marker expression levels. If the chondroitin produces a sample profile that matches the reference profile and thus indicative of responsiveness to the chondroitin, the chondroitin is then administered to the patient to improve cartilage growth or cartilage maintenance. If the hyaluronic acid produces a sample profile that matches the reference profile and thus indicative of responsiveness to the hyaluronic acid, the hyaluronic acid is then administered to the patient to improve cartilage growth or cartilage maintenance. Alternatively, the chondroitin or hyaluronic acid can be custom compounded to form a custom supplement and administered to the patient.

### Example 2

Synovial mesenchymal stem cells are isolated from a patient and cultured in an incubator at 37° C. and 5% CO₂. Each of glucosamine and/or collagen is diluted to a physiologically appropriate concentration, about 500 to about 900 ng/ml, in the stem cell culture medium. After culturing stem cell samples in the presence or absence of each agent, FACS is used to determine if the cells cultured in the presence of any one of the agents were being moved toward chondrogenesis. Exemplary markers used to make this determination include CD90⁻, CXCL1⁺, F1T3L⁺, CCR3⁺, and CCR4⁺. The expression levels of the various markers are used to create a sample profile. The sample profile generated by treatment of the stem cells is compared to a reference profile, or a profile of desirable marker expression levels. If the glucosamine produces a sample profile that matches the reference profile and thus indicative of responsiveness to the glucosamine, the glucosamine is then administered to the patient to improve cartilage growth or cartilage maintenance. If the collagen produces a sample profile that matches the reference profile and thus indicative of responsiveness to the collagen, the collagen is then administered to the patient to improve cartilage growth or cartilage maintenance. Alternatively, the glucosamine or collagen can be custom compounded to form a custom supplement and administered to the patient.

### Example 3

Synovial mesenchymal stem cells are isolated and cultured in an incubator at 37° C. and 5% CO₂. Each of one or more NSAIDS (non-steroidal anti-inflammatory drugs), for example, ketoprofen, is diluted to a physiologically appropriate concentration, about 10 to about 20 ng/ml, in the stem cell culture medium. After culturing stem cell samples in the presence or absence of the one or more NSAIDs, FACS is used to determine if the cells cultured in the presence of any one of the agents were being moved toward chondrogenesis. Exemplary markers used to make this determination include CD90⁻, CXCL1⁺, F1T3L⁺, CCR3⁺, and CCR4⁺. The expression levels of the various markers are used to create a sample profile. The sample profile generated by treatment of the stem cells is compared to a reference profile, or a profile of desirable marker expression levels. If ketoprofen produces a sample profile that matches the reference profile and thus indicative of responsiveness to the ketoprofen, the ketoprofen is then administered to the patient to improve cartilage growth or cartilage maintenance. Alternatively, the ketoprofen (or other NSAID to which the patient is responsive) can be custom compounded to form a custom supplement and administered to the patient.

### Example 4

Epidermal stem cells are isolated from a patient with aging skin and cultured in an incubator at 37° C. and 5% CO₂. Pinocembrin, a Lippia origanoides plant extract, is diluted to a physiologically appropriate concentration, about 1 to about 200 ng/ml, in the stem cell culture medium. After culturing stem cell samples in the presence or absence of the pinocembrin, Northern blot analysis is used to determine if the cells cultured in the presence of the extract increased production of hyaluronan, a substance excreted by dermal fibroblasts to moisturize and restore skin elasticity. The sample profile generated by treatment of the epidermal stem cells is compared to a reference profile, or a profile of desirable marker expression levels. If pinocembrin produces a sample profile that matches the reference profile and thus indicative of improved skin elasticity, the pinocembrin is then administered topically to the patient to reduce effects of aging. Alternatively, the pinocembrin can be custom compounded to form a custom supplement and administered to the patient.

### Example 5

Hematopoietic stem cells are isolated from a patient suffering from fibromyalgia and cultured in an incubator at 37° C. and 5% CO₂. Duloxetine is diluted to a physiologically appropriate concentration, about 60 to about 120 ng/ml, in the stem cell culture medium. After culturing stem cell samples in the presence or absence of duloxetine, microarray is used to generate a sample profile. The sample profile generated by treatment of the mesenchymal stem cells is compared to a reference profile obtained by pooling microarray results obtained from stem cell samples of several fibromyalgia patients successfully treated with duloxetine. If duloxetine produces a sample profile that matches the reference profile and thus indicative pain relief and/or improved sleeping habits, the compound is then administered to the patient to treat fibromyalgia. Alternatively, the duloxetine can be custom compounded to form a custom supplement and administered to the patient.

The description of the present invention has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiment described and shown in the figures was chosen and described in order to best explain the principles of the invention, the practical application, and to enable others of ordinary skill in the art to understand the invention for various embodiments with various modifications as are suited to the particular use contemplated.

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

What is claimed is:

1. A method of assessing whether a subject having a condition, disorder, dysfunction, or disease would respond to treatment with an agent, the method comprising:
obtaining a stem cell sample from the subject;
culturing the sample;
exposing the sample to an agent;
evaluating the sample in the presence of and in the absence of the agent to create a sample profile;
comparing the sample profile to a reference profile indicative of responsiveness to the agent;
wherein the similarity between the sample profile and the reference profile indicates the subject's responsiveness to the agent.

2. The method of claim 1, wherein the stem cell sample comprises embryonic stem cells, fetal stem cells, umbilical cord stem cells, placental derived stem cells, very small embryonic-like stem cells, or adult stem cells of hematopoietic, mesenchymal, epidermal, or endodermal lineages.

3. The method of claim 1, wherein the agent is selected from the group consisting of nutritional supplements, nutraceuticals, growth factors, cytokines, hormones, essential nutrients, registered chemicals, novel chemicals, biologicals, and environmental reagents.

4. The method of claim 1, wherein the step of evaluating comprises measuring a cellular response.

5. The method of claim 4, wherein the cellular response is selected from the group consisting of transcription, expression, protein activation, protein inactivation, proliferation, differentiation, apoptosis, and alteration in morphology.

6. The method of claim 5, wherein the step of evaluating comprises measuring transcription using a microarray.

7. The method of claim 1, wherein the reference profile is generated by evaluating one or more samples in the presence of and absence of one or more agents typically used to treat the condition, disorder, dysfunction, or disease.

8. The method of claim 7, wherein the condition, disorder, dysfunction, or disease is selected from the group consisting of:
a) the degenerative conditions of aging selected from the group consisting of osteoarthritis, coronary heart disease, and osteoporosis;
b) disorders of the immune system selected from the group consisting of lupus, systemic lupus erythematosus, multiple sclerosis, ankylosing spondylitis, scleroderma, and rheumatoid arthritis;
c) disorders of the neurologic system selected from the group consisting of Alzheimer's disease, Parkinson's, and spinal cord injury;
d) disorders of gastrointestinal system selected from the group consisting of irritable bowel syndrome, gastroesophageal reflux disease, Crohn's disease, and ulcerative colitis; and
e) disorders of the musculo skeletal system selected from the group consisting of cartilage injuries, tendonopathies, fibromyalgia, ligament injuries, degenerative disc disease, and sciatica.

9. A method of providing a custom treatment for a subject, the method comprising:
obtaining a stem cell sample from the subject;
culturing the sample;
exposing the sample to an agent;
evaluating the sample in the presence of and in the absence of the agent to create a first sample profile;
comparing the first sample profile to a first reference profile indicative of non-responsiveness to the agent, wherein the similarity between the first sample profile and the first reference profile indicates the subject's non-responsiveness to the agent;
designing a custom compound and exposing the sample to the custom compound;
evaluating the sample in the presence of and in the absence of the custom compound to create a second sample profile;
comparing the second sample profile to a second reference profile indicative of responsiveness to the agent;
wherein the similarity between the second sample profile and the second reference profile indicates the subject's responsiveness to the custom compound; and
wherein the custom compound is provided to the subject as a custom treatment.

10. The method of claim 9, wherein the custom compound comprises nutritional supplements, nutraceuticals, growth factors, cytokines, hormones, essential nutrients, registered chemicals, novel chemicals, biologicals, and environmental reagents.

11. The method of claim 9, wherein the subject has altered chondrogenesis, osteogenesis, altered neurologic function or altered fibrogenesis.

12. A method of screening a custom compound for efficacy, safety, or lack of adverse effect, the method comprising:
obtaining a stem cell sample from a subject;
culturing the sample;
exposing the sample to a custom compound;
evaluating the sample in the presence of and in the absence of the custom compound to create a sample profile;
comparing the sample profile to a reference profile generated by an agent which exhibits efficacy, safety, or lack of adverse effect;
wherein the similarity between the sample profile and the reference profile indicates the efficacy, safety, or lack of adverse effect of the custom compound.

13. The method of claim 12, wherein the method further comprises the step of comparing the sample profile to a second reference profile generated by a second agent which exhibits an adverse effect.

14. The method of claim 12, wherein the method further comprises the step of comparing the sample profile to a second reference profile generated by a second agent which exhibits undesired efficacy.

15. The method of claim 12, wherein the method further comprises the step of comparing the sample profile to a second reference profile generated by a second agent which is unsafe.

16. A method of treating a subject in need thereof, the method comprising:
obtaining a stem cell sample from the subject;
culturing the sample;
exposing the sample to an agent;
evaluating the sample in the presence of and in the absence of the agent to create a first sample profile;
comparing the first sample profile to a first reference profile indicative of non-responsiveness to the agent, wherein the similarity between the first sample profile and the first reference profile indicates the subject's non-responsiveness to the agent;
designing a custom compound and exposing the sample to the custom compound;
evaluating the sample in the presence of and in the absence of the custom compound to create a second sample profile;
comparing the second sample profile to a second reference profile indicative of responsiveness to the agent;
wherein the similarity between the second sample profile and the second reference profile indicates the subject's responsiveness to the custom compound; and
wherein the custom compound is provided to the subject as a custom treatment.

17. A method of treating a subject in need thereof, the method comprising:
obtaining a stem cell sample from the subject;
culturing the sample;
exposing the sample to an agent;
evaluating the sample in the presence of and in the absence of the agent to create a sample profile;
comparing the sample profile to a reference profile indicative of responsiveness to the agent; wherein the similarity between the sample profile and the reference profile indicates the subject's responsiveness to the agent; and
treating the subject with the agent.

18. The method of claim 17, wherein the subject suffers from:
a) degenerative conditions of aging selected from the group consisting of osteoarthritis, coronary heart disease, and osteoporosis;
b) disorders of the immune system selected from the group consisting of lupus, systemic lupus erythematosus, multiple sclerosis, ankylosing spondylitis, scleroderma, and rheumatoid arthritis;
c) disorders of the neurologic system selected from the group consisting of Alzheimer's disease, Parkinson's, and spinal cord injury;
d) disorders of gastrointestinal system selected from the group consisting of irritable bowel syndrome, gastroesophageal reflux disease, Crohn's disease, and ulcerative colitis; and
e) disorders of the musculo skeletal system selected from the group consisting of cartilage injuries, tendonopathies, fibromyalgia, ligament injuries, degenerative disc disease, and sciatica.

19. The method of claim 17, wherein the reference profile is generated by evaluating one or more samples in the presence of and absence of one or more agents used to treat:
a) degenerative conditions of aging selected from the group consisting of osteoarthritis, coronary heart disease, and osteoporosis;
b) disorders of the immune system selected from the group consisting of lupus, systemic lupus erythematosus, multiple sclerosis, ankylosing spondylitis, scleroderma, and rheumatoid arthritis;
c) disorders of the neurologic system selected from the group consisting of Alzheimer's disease, Parkinson's, and spinal cord injury;
d) disorders of gastrointestinal system selected from the group consisting of irritable bowel syndrome, gastroesophageal reflux disease, Crohn's disease, and ulcerative colitis; and
e) disorders of the musculo skeletal system selected from the group consisting of cartilage injuries, tendonopathies, fibromyalgia, ligament injuries, degenerative disc disease, and sciatica.

20. A computer-aided system comprising:
(a) a computer readable medium comprising computer-executable instructions configured to predict responsiveness of a subject to treatment with an agent;
(b) input data from: (i) a subject profile; and (ii) a reference profile; and
(c) an algorithm capable of comparing the data; wherein the system outputs a predicted responsiveness by the subject to the agent.

21. The method of claim 9, wherein the custom compound is provided to the patient formulated as a cosmetic.

22. The method of claim 17, wherein the agent is provided to the patient formulated as a cosmetic.
